(19) United States
(12) Patent Application Publication
(10) Pub. No.: US 2024/0108816 A1
(43) Pub. Date: Apr. 4, 2024

(54) Injection Devices

(71) Applicant: Joseph R. ROBINSON, New Rochelle, NY (US)

(72) Inventor: Joseph R. ROBINSON, New Rochelle, NY (US)

(21) Appl. No.: 18/376,256

(22) Filed: Oct. 3, 2023

Related U.S. Application Data
(60) Provisional application No. 63/412,663, filed on Oct. 3, 2022.

(51) Int. Cl. A61M 5/32; A61M 5/24; A61M 5/31

(52) U.S. Cl. CPC A61M 5/3245; A61M 5/24; A61M 5/3137; A61M 2202/0007

(57) ABSTRACT

The present invention is directed to syringes that can be manufactured by blow molding and can be aseptically filled and finished, i.e., Blow-Fill-Seal (“BFS”). The present invention is also directed to pen-type injectors that accommodate cartridges that can be manufactured by BFS technology.

### CROSS-REFERENCE TO RELATED APPLICATIONS

This application claims the benefit of, and priority to U.S. Provisional Application No. 63/412,663, filed on Oct. 3, 2022. The entire contents of this application is incorporated herein by reference in its entirety.

### FIELD OF THE INVENTION

The invention relates to syringes used for injecting liquids or gels, such as, for example, pharmaceutical and biologics formulations and vaccines, into tissues. The invention provides a versatile syringe that can be made to contain a single dose of a liquid, gel, gas, or formulation, that can be non-reusable, and that can be aseptically manufactured, filled, and finished.

The invention also relates to pen type injectors used for injecting liquids or gels into tissues. The invention provides injectors that can inject doses of a liquid, gel, gas, or formulation. The injectors are reusable, but the cartridges for the injector preferably are not.

### BACKGROUND OF THE INVENTION

A conventional syringe consists of a plunger that fits tightly within a rigid, non-compressible cylindrical tube called a barrel. The plunger can be linearly pulled and pushed along the inside of the tube, allowing the syringe to take in and expel liquid or gas through a discharge orifice at the open end of the tube. The open end of the syringe may be fitted with a hypodermic needle, a nozzle, or tubing to direct the flow of a liquid, gel, gas, or formulation, into and out of the barrel. Syringes are frequently used to administer medicinal injections from vials or to infuse intravenous therapy.

Many liquid and gel formulations are administered parenterally through syringes. Syringe use is growing rapidly due to the rising prevalence of chronic illnesses, such as diabetes, rheumatoid arthritis, and other autoimmune diseases and due to the general surging demand for biological drugs.

A trained medical professional must follow multiple steps to prepare an injection from a vial and disposable syringe. This is cumbersome and susceptible to dosing and medication selection errors. This is often difficult or inconvenient for a chronically ill patient or a patient with limited dexterity to do at home. Furthermore, current plastic syringes are liable to dosage inaccuracies because plastic is expandable.

A prefilled syringe typically consists of a barrel, an internal plunger within in the barrel, a plunger rod, and a needle attachment. However, fluid is not drawn up manually into prefilled syringe barrels when the injection is to be administered. Instead, the syringe is filled with the drug formulation during the sterile manufacturing process. The plunger and plunger rod are also inserted into the barrel during this manufacturing process.

Prefilled syringes enhance flexibility and convenience. They are also safer, as reduced complexity in the administration process lowers the risk of dosing errors and contamination. Furthermore, the wrong drug cannot be accidently loaded into the syringe by the administrator. Prefilled syringes also accelerate vaccination programs and eliminate waste inherent in the use of multi-dose vials.

Prefilled syringes and injector pens can be used in many different applications and settings including at home for self-administration to treat chronic illnesses, in hospitals, in nursing homes, in doctors' offices, and in specialist clinics.

A pen-type injector is a device used for injecting medication. Injector pens make injectable medication easier and more convenient, thus increasing patient adherence. Injector pens are easier to use by untrained people and by people with low dexterity or poor vision, or by people who need portability to administer medicine on time. Injector pens also decrease the fear or adversity towards self-injection of medications, which increases the likelihood that a person will take the medication.

Injector pens are commonly used for medications that are injected repeatedly by a person. Many medications are available as injector pens. Surveys have shown that a vast majority of people would prefer an injector pen over vial and syringe administration if one was available.

Many drugs have been approved in combination with prefilled syringes or injector pens across different geographies, including North America, Europe, and Asia-Pacific. Moreover, many clinical-stage drugs are being evaluated in combination with prefilled syringes or injector pens across different phases of development. The demand for prefilled syringe and injector pen dosage forms is rising, but their production is complicated and time consuming.

Pharmaceutical manufacturing can be broken down into two main processes—therapeutic product production and therapeutic product packaging. The latter is termed “fill/finish”. Fill/finish is the final step in the manufacturing process and is among the most crucial stages of drug product manufacturing. Many pharmaceutical products need to be aseptically (i.e., sterilely) fill/finished, whether for clinical study use or commercial sale.

Traditionally manufactured pre-filled syringes have been difficult to fill in volume because of the structure of the syringe.

BFS technology is an aseptic fill/finish manufacturing technique used to produce small (0.1 mL) through large (over 500 mL) volume liquid- or gel-filled containers in a single operation. Adapting BFS to fill/finish syringes and injector pens, and particularly, prefilled syringes, has been difficult due to the structure of traditional syringes.

There is a need for prefilled syringes, injector pens, and injector pen components that can be inexpensively and aseptically manufactured so that health care and treatment costs can be minimized without diminution of quality of care. There is a need for increased safety and ease of use of prefilled syringes, injector pens, and injector pen components, particularly to increase patient compliance and adherence. There is a need for prefilled syringes, injector pens, and injector pen components that maximize the ability to mobilize, effectively use, and eliminate waste of new medications and vaccines. There is a need for prefilled syringes, injector pens, and injector pen components that can rapidly be deployed in large quantities.

The present inventor has surprisingly and unexpectedly solved these problems, needs, and more with the presently claimed syringes, injector pens, components, and methods. The present invention is easily adaptable to different fill a wide range of amounts or dosages while maintaining high speed.

### SUMMARY OF THE INVENTION

The present invention provides a syringe comprising:
(a) a one piece body, the body comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a hollow tube piercing end and a discharge end;
      the distal end being pierceable by the needle hollow tube piercing end of the needle to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue; and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, preferably, compress radially, (i.e., inwardly collapse) the hollow tubular section so that when a needle containing piece is piercing the distal end of the hollow tubular section, at least a portion of any contents of the hollow tubular section passes through the needle discharge end.

The present invention also provides a prefilled syringe comprising:
(a) a one piece body, the body comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a hollow tube piercing end and a discharge end;
      the distal end being pierceable by the needle hollow tube piercing end of the needle to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue and wherein said hollow tubular section contains a therapeutically effective amount (preferably, a single dose) of a pharmaceutical or biological drug or vaccine (which may be aseptically sealed therein); and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, preferably, compress radially, (i.e., inwardly collapse) the hollow tubular section so that when a needle containing piece is piercing the distal end of the hollow tubular section, at least a portion of any contents of the hollow tubular section passes through the needle discharge end.

The present invention also provides a device comprising the syringe of the present invention and a needle containing piece.

The present invention also provides a kit comprising a strip of a plurality of prefilled syringes, wherein the syringes can be separated from the strip one at a time.

The present invention also provides a method of injecting a liquid or gel (preferably, a therapeutically effective amount of a pharmaceutical or biological drug or vaccine) into an animal in need of the liquid or gel with the syringe described above, the method comprising:
(a) providing a syringe of the present invention with the liquid or gel in the hollow tubular section;
(b) placing on the syringe, a needle containing piece, the needle containing piece comprising a retainer and a needle, the needle having a hollow tube piercing end and a discharge end, whereby the hollow tube piercing end of the needle pierces the distal end of the tubular section;
(c) inserting the needle discharge end of the needle through at least a portion of one or more tissues of the animal; and
(d) sliding the slideable collar longitudinally along at least a portion of the outside of the hollow tubular section, thereby inwardly compressing at least a portion of the hollow tubular section to force at least a portion of the liquid or gel (preferably, a therapeutically effective amount of a pharmaceutical or biological drug or vaccine) through the needle discharge end and into the tissue.

The present invention also provides a syringe comprising:
(a) a one piece body, the body comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a a discharge end;
      at least a portion of the distal end being permanently openable, or openable and closeable, preferably by fracture, rupture, or valve, when a slideable collar is moved longitudinally along said hollow tubular section, preferably by pressure generated by the movement of said slideable collar on any contents of said hollow tubular section, to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue; and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, preferably, compress radially, (i.e., inwardly collapse) the hollow tubular section so that when a needle containing piece is attached the distal end of the hollow tubular section and said at least a portion of said distal end of said hollow tubular section is open, at least a portion of any contents of the hollow tubular section passes through the needle discharge end.

The present invention also provides a prefilled syringe comprising:
(a) a one piece body, the body comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a a discharge end;
      at least a portion of the distal end being permanently openable, or openable and closeable, preferably by fracture, rupture, or valve, when a slideable collar is moved longitudinally along said hollow tubular section, preferably by pressure generated by the movement of said slideable collar on any contents of said hollow tubular section, to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue and wherein said hollow tubular section contains a therapeutically effective amount (preferably, a single dose) of a pharmaceutical or biological drug or vaccine (which may be aseptically sealed therein); and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, preferably, compress radially, (i.e., inwardly collapse) the hollow tubular section so that when a needle containing piece is attached the distal end of the hollow tubular section and said at least a portion of said distal end of said hollow tubular section is open, at least a portion of any contents of the hollow tubular section passes through the needle discharge end.

The present invention also provides a method of injecting a liquid or gel (preferably, a therapeutically effective amount of a pharmaceutical or biological drug or vaccine) into an animal in need of the liquid or gel with the syringe described above, the method comprising:
(a) providing a syringe of the present invention with the liquid or gel in the hollow tubular section;
(b) placing on the syringe, a needle containing piece, the needle containing piece comprising a retainer and a needle;
(c) inserting the needle discharge end of the needle through at least a portion of one or more tissues of the animal; and
(d) sliding the slideable collar longitudinally along at least a portion of the outside of the hollow tubular section, thereby opening at least a portion of the distal end of the hollow tubular section inwardly and compressing at least a portion of the hollow tubular section to force at least a portion of the liquid or gel (preferably, a therapeutically effective amount of a pharmaceutical or biological drug or vaccine and, most preferably, a single dose) through the needle discharge end and into the tissue.

The present invention also provides an injector pen comprising:
(a) an outer body having a lower cartridge receiving area, an upper slideable collar mechanism area, and a needle containing piece receiver;
(b) a cartridge in the lower cartridge receiving area, the cartridge comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the hollow tubular section and said distal end being pierceable by a needle of a needle containing piece to provide for open communication from the inside of the cartridge for transport of any contents of the hollow tubular section through a needle the needle discharge end of the needle containing piece;
   (ii) an extension piece extending longitudinally from the proximal end of said hollow tubular section and in a direction opposite and away from the hollow tubular section; wherein said hollow tubular section does not collapse, flatten, or compress longitudinally in an amount sufficient to eject more than an insignificant amount of the formulation from said cartridge before the entire dose of the cartridge delivered to tissue; and
   (iii) a therapeutically effective amount (preferably, a single dose) of a pharmaceutical or biological drug or vaccine (which may be aseptically sealed therein);
(c) a slideable collar in the upper area, the slideable collar comprising a compression portion, the compression portion being adapted to slide longitudinally along longitudinal flanges and outer surface of the cartridge to flatten the cartridge, one or more extension arms extending from the compression piece in a longitudinal direction of the injector pen opposite and away from the cartridge receiving area opposite from the compression piece; a bridge connecting the extension arms at their respective ends opposite the compression portion, and a user activated slideable collar driver; and
(d) a needle containing piece on the needle containing piece receiver, said needle containing piece comprising a retainer and a needle, the needle having a hollow tube piercing end and a discharge end.

The present invention also provides a method of injecting a liquid or gel into an animal in need of said liquid or gel, said method comprising:
(a) providing the foregoing injector pen;
(b) placing said needle discharge end into a tissue of said animal; and
(c) activating said slideable collar to move longitudinally along at least a portion of the outside of said cartridge, thereby flattening at least a portion of said hollow tubular section of said cartridge to force said therapeutically effective amount (preferably a single dose) of a pharmaceutical or biological drug or vaccine through said needle discharge end and into said tissue.

The present invention also provides an injector pen comprising:
(a) an outer body having a lower cartridge receiving area, an upper slideable collar mechanism area, and a needle containing piece receiving area below said cartridge receiving area;
(b) a cartridge in said lower cartridge receiving area, said cartridge comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, said hollow tubular section having a closed proximal end, a closed distal end, and at least one flange extending along at least a majority of the length of said hollow tubular section and said distal end being pierceable by a needle of a needle containing piece to provide for open communication from the inside of said cartridge for transport of any contents of said hollow tubular section through a needle the needle discharge end of said needle containing piece; wherein said hollow tubular section does not collapse, flatten, or compress longitudinally in an amount sufficient to eject more than an insignificant amount of the formulation from the syringe before the entire dose of the syringe is delivered to tissue;
   (ii) a needle receiving section at said distal end,
   (iii) an extension piece extending longitudinally from said proximal end of said hollow tubular section and in a direction opposite said hollow tubular section, and
   (iv) a therapeutically effective amount (preferably, a single dose) of a pharmaceutical or biological drug or vaccine (which may be aseptically sealed therein) contained in said hollow tubular section;
(c) a slideable collar in said upper area, said slideable collar comprising a compression portion, said compression portion being adapted to slide longitudinally along longitudinal flanges and outer surface of said cartridge to flatten said cartridge, one or more extension arms extending in a longitudinal direction of said injector pen away from said cartridge receiving area opposite from said compression piece; a bridge connecting said extension arms at their respective ends opposite said compression portion, and a user activated slideable collar driver, wherein at least a portion of said extension piece of said cartridge is inserted into said slideable collar; and
(d) a needle containing piece in said needle piece receiving, said needle containing piece being proximate to said needle receiving section of said cartridge, said needle containing piece comprising a needle having a hollow tube piercing end and a discharge end;
whereby when said slideable collar moves longitudinally toward said tubular section of said cartridge, said cartridge needle receiving section engages said needle containing piece and said hollow tube piercing section pierces said distal end of said cartridge thereby opening communication between any contents of said cartridge and said needle, whereby when said slideable collar moves further longitudinally toward said tubular section of said cartridge said needle protrudes from said device in an amount sufficient to penetrate tissue of an animal in need of such formulation, and when slideable collar moves longitudinally over said hollow tubular section, said therapeutically effective amount (preferably, a single dose) of a pharmaceutical or biological drug or vaccine is injected into said tissue.

The present invention further provides a method of injecting a liquid or gel into an animal in need of said liquid or gel, the method comprising:
(a) providing the foregoing injector pen;
(b) placing said injector pen against said animal; and
(d) actuating said injector pen to move slideable collar longitudinally toward said tubular section of said cartridge, said cartridge needle receiving section engaging said needle containing piece and said hollow tube piercing section piercing said distal end of said cartridge thereby opening communication between any contents of said cartridge and said needle, further to move said slideable collar further longitudinally toward said tubular section of said cartridge to move said needle to protrude from said device in an amount sufficient to penetrate tissue of an animal in need of such formulation, and further to move said slideable collar longitudinally over said hollow tubular section to inject said therapeutically effective amount (preferably, a single dose) of a pharmaceutical or biological drug or vaccine into said tissue from said needle discharging end.

The present invention also provides an injector pen comprising:
(a) an outer body having a lower cartridge receiving area, an upper slideable collar mechanism area, and a needle containing piece receiver;
(b) a cartridge in the lower cartridge receiving area, the cartridge comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, the hollow tubular section having a closed proximal end, a closed distal end, and at least one flange extending along at least a majority of the length of the hollow tubular section and the distal end adapted to receive and secure a needle containing piece, the needle containing piece comprising a retainer and a needle, the needle having a discharge end;
      at least a portion of the distal end being permanently openable, or openable and closeable, preferably by fracture, rupture, or valve, when a slideable collar is moved longitudinally along said hollow tubular section, preferably by pressure generated by the movement of said slideable collar on any contents of said hollow tubular section, to provide for open communication from the inside of the hollow tubular section for transport of any contents of the hollow tubular section through the needle discharge end;
   (ii) an extension piece extending longitudinally from the proximal end of said hollow tubular section and in a direction opposite and away from the hollow tubular section; wherein said hollow tubular section does not collapse, flatten, or compress longitudinally in an amount sufficient to eject more than an insignificant amount of the formulation from said cartridge before the entire dose of the cartridge delivered to tissue; and
   (iii) and wherein said hollow tubular section contains a therapeutically effective amount (preferably, a single dose) of a pharmaceutical or biological drug or vaccine (which may be aseptically sealed therein);
(c) a slideable collar in the upper area, the slideable collar comprising a compression portion, the compression portion being adapted to slide longitudinally along longitudinal flanges and outer surface of the cartridge to flatten the cartridge, one or more extension arms extending from the compression piece in a longitudinal direction of the injector pen opposite and away from the cartridge receiving area opposite from the compression piece; a bridge connecting the extension arms at their respective ends opposite the compression portion, and a user activated slideable collar driver; and
(d) a needle containing piece on the needle containing piece receiver, said needle containing piece comprising a retainer and a needle, the needle having a discharge end.

The present invention also provides a method of injecting a liquid or gel into an animal in need of said liquid or gel, said method comprising:
(a) providing the foregoing injector pen;
(b) placing said needle discharge end into a tissue of said animal; and
(c) activating said slideable collar to move longitudinally along at least a portion of the outside of said cartridge, thereby opening at least a portion of the distal end of the hollow tubular section inwardly and flattening at least a portion of said hollow tubular section of said cartridge to force said therapeutically effective amount (preferably, a single dose) of a pharmaceutical or biological drug or vaccine through said needle discharge end and into said tissue.

The present invention also provides an injector pen comprising:
(a) an outer body having a lower cartridge receiving area, an upper slideable collar mechanism area, and a needle containing piece receiving area below said cartridge receiving area;
(b) a cartridge in said lower cartridge receiving area, said cartridge comprising:
   (i) a hollow tubular section at least partially and preferably substantially completely non-longitudinally flattenable (i.e., inwardly collapsible or compressible, preferably, at least partially radially collapsible or compressible) under direct or indirect force from a slideable collar, said hollow tubular section having a closed proximal end, a closed distal end, and at least one flange extending along at least a majority of the length of said hollow tubular section and the distal end adapted to receive and secure a needle containing piece, the needle containing piece comprising a retainer and a needle, the needle having a discharge end;
      at least a portion of the distal end being permanently openable, or openable and closeable, preferably by fracture, rupture, or valve, when a slideable collar is moved longitudinally along said hollow tubular section, preferably by pressure generated by the movement of said slideable collar on any contents of said hollow tubular section, to provide for open communication from the inside of the hollow tubular section for transport of any contents of the hollow tubular section through the needle discharge end;
      wherein said hollow tubular section does not collapse, flatten, or compress longitudinally in an amount sufficient to eject more than an insignificant amount of the formulation from the syringe before the entire dose of the syringe is delivered to tissue;
   (ii) a needle receiving section at said distal end,
   (iii) an extension piece extending longitudinally from said proximal end of said hollow tubular section and in a direction opposite said hollow tubular section, and
   (iv) a therapeutically effective amount (preferably, a single dose) of a pharmaceutical or biological drug or vaccine contained in said hollow tubular section;
(c) a slideable collar in said upper area, said slideable collar comprising a compression portion, said compression portion being adapted to slide longitudinally along longitudinal flanges and outer surface of said cartridge to flatten said cartridge, one or more extension arms extending in a longitudinal direction of said injector pen away from said cartridge receiving area opposite from said compression piece; a bridge connecting said extension arms at their respective ends opposite said compression portion, and a user activated slideable collar driver, wherein at least a portion of said extension piece of said cartridge is inserted into said slideable collar; and
(d) a needle containing piece in said needle piece receiving, said needle containing piece being proximate to said needle receiving section of said cartridge, said needle containing piece comprising a discharge end;
whereby when said slideable collar moves longitudinally toward said tubular section of said cartridge, said cartridge needle receiving section engages said needle containing piece and said at least a portion of the distal end opens thereby opening communication between any contents of said cartridge and said needle, whereby when said slideable collar moves further longitudinally toward said tubular section of said cartridge said needle protrudes from said device in an amount sufficient to penetrate tissue of an animal in need of such formulation, and when slideable collar moves longitudinally over said hollow tubular section, said therapeutically effective amount (preferably, a single dose) of a pharmaceutical or biological drug or vaccine is injected through the needle discharge end and into said tissue.

The present invention further provides a method of injecting a liquid or gel into an animal in need of said liquid or gel, the method comprising:
(a) providing the foregoing injector pen;
(b) placing said injector pen against said animal; and
(c) actuating said injector pen to move the slideable collar longitudinally toward said tubular section of said cartridge, said cartridge needle receiving section engaging said needle containing piece and at least a portion of said distal end of said hollow tube opening, thereby opening communication between any contents of said cartridge and said needle, and further to move said slideable collar further longitudinally toward said tubular section of said cartridge to move said needle to protrude from said device in an amount sufficient to penetrate tissue of an animal in need of such formulation, and further to move said slideable collar longitudinally over said hollow tubular section to inject said therapeutically effective amount (preferably, a single dose) of a pharmaceutical or biological drug or vaccine into said tissue from said needle discharging end.

The present invention also provides a blow fill machine and a blow mold adapted to manufacture the syringes and cartridges of the present invention.

### BRIEF DESCRIPTION OF THE DRAWINGS

FIG. 1 is a cross-sectional view of one embodiment of a syringe of the present invention.

FIG. 2 is a cross-sectional view of one embodiment of a multi-chamber hollow tubular section of a syringe of the present invention.

FIG. 3 is a cross-sectional view of another embodiment of a multi-chamber hollow tubular section of a syringe of the present invention.

FIG. 4 is a cross-sectional view of one embodiment of an injector pen of the present invention.

FIG. 5 is a cross-sectional view of another embodiment of an injector pen of the present invention.

FIG. 6 is a cross-sectional view of a portion above the needle attachment area of a further embodiment of an injector pen of the present invention.

FIG. 7 is a cross-sectional view of another embodiment of an injector pen of the present invention.

### DETAILED DESCRIPTION OF THE INVENTION

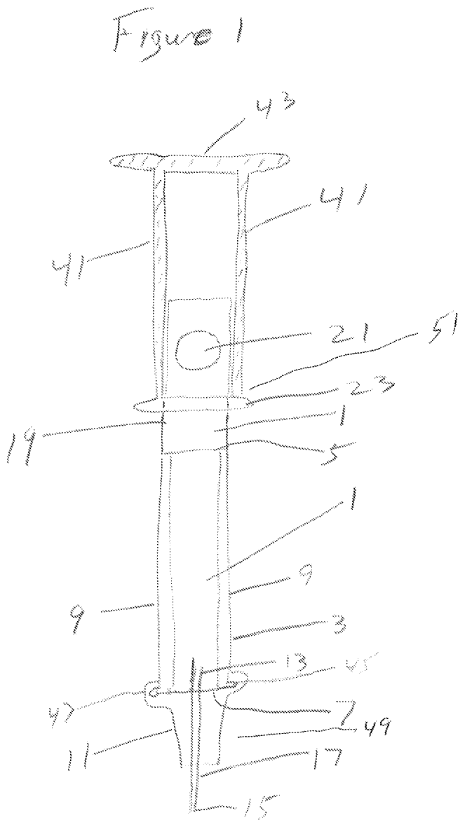

Referring to FIG. 1, the syringe of the present invention includes a one piece body (1). The body includes a hollow flattenable or inwardly compressible tubular section (3). The hollow tubular section is compressible inwardly (preferably in a radial direction) along a majority of its longitudinal length to flatten at least a majority of the tube while maintaining the longitudinal length of the tube and, preferably, along its longitudinal length sufficient to empty substantially all (e.g., a therapeutically effective amount of the dose) of any liquid or gel contents from the hollow tubular section. Preferably, once the hollow tubular section has been flattened, it need not or does not substantially rebound to a non-flattened/compressed state. The hollow tubular section is preferably not compressible, flattenable, or collapsible in the longitudinal direction in any amount sufficient to assist in evacuating liquid or gel from the hollow tubular section. Preferably, the syringe, and particularly, the hollow tubular section (particularly in the longitudinal direction), is of sufficient rigidity (particularly longitudinal rigidity) so that a full cartridge does not collapse, flatten, or compress longitudinally in an amount that adversely affects or impedes injection of the syringe contents or in an amount sufficient to eject more than an insignificant amount of the formulation from the syringe before the entire dose of the cartridge is delivered to the tissue by the inward flattening of the hollow tubular section. An insignificant amount is an amount that does not adversely affect the therapeutic efficacy of the amount of the formulation in the full syringe. Preferably, the syringe is also of sufficient rigidity in the longitudinal direction to facilitate or accommodate insertion of a needle containing piece into the syringe and into tissue. These longitudinal rigidity conditions are applicable to a full syringe but not necessarily applicable to a syringe after its contents have been injected into a tissue.

This tubular section has a closed proximal end (5), a closed distal end (7), and at least one longitudinal flange (9) extending along at least a majority of the length of the tubular section. Preferably, flanges extend longitudinally along the entire tubular section. The flange(s) may also include a guide ridge or rail or the like in the longitudinal direction along at least a portion of the flange that will accept and guide the slideable collar to pass over and flatten the tube. The proximal and distal ends of the tubular section are sealed in order to contain a liquid or gel in the hollow tubular section that will be injected by the syringe.

Preferably, the syringe body is not substantially compressible in the longitudinal direction. Most preferably, it is substantially smooth without folds like a bellows that would allow it to compress longitudinally, although it may have non-collapsing longitudinal ridges for strength.

The horizontal cross-sectional shape of the hollow tubular section of the syringes and cartridges of the present invention may be of any suitable shape such as, for example, circular, oval, square, square with rounded corners, rectangular, rectangular with rounded corners, trapezoidal, trapezoidal with rounded corners, and the like. The shape may vary across the hollow tubular sections length. It may be flat or another shape along a portion of its length or entirety. Preferably, the hollow tubular section will taper towards its distal end, it proximal end, or both, for example, as would be formed in heat sealing a plastic.

The hollow tubular section can form a single chamber or a plurality of chambers or compartments. The plurality of chambers can be arranged linearly one after the other (e.g., in tandem), parallel to one another, or in a combination thereof. There could be a breakable or breachable barrier or connection between the chambers. Preferably, the barrier or connection would be broken, opened, or breached by pressure directly or indirectly from the slideable collar (e.g., the slideable collar is moved along the tubular section sufficiently to force the contents of the upper chamber to break, open, or breach the barrier or connection between the two chambers with the contents of the upper chamber then emptying into the lower chamber. A multi-chamber tubular section could allow for the administration of more than one liquid or gel in a single injection or the mixing of multiple components (at least one being a liquid or gel) in the syringe itself before or during injection. This would be particularly suitable for lyophilized formulation that must be reconstituted with a diluent before administration, wherein the upper most chamber contains the diluent and the lower chamber contains the lyophilized product. Adding the diluent to the lyophized product will form a liquid for injection.

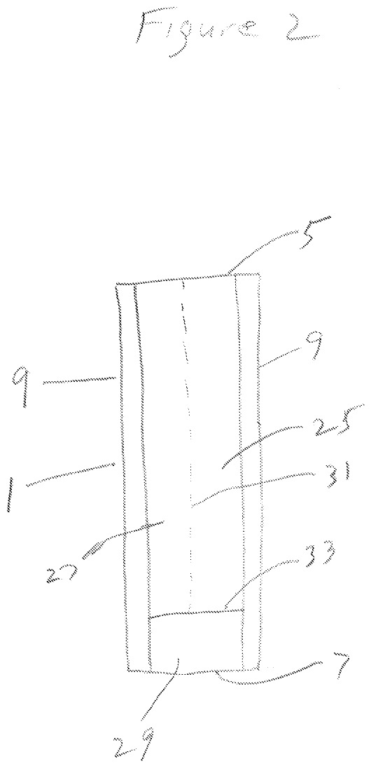

FIG. 2 illustrates a multi-chamber hollow tubular section in which there are two longitudinal parallel chambers (25), (27) separated from one another by an internal barrier that is non-breakable. A mixing chamber (29) is located to receive the contents of longitudinal chambers (25) and (27) through a breakable or breachable barrier (33). When the contents of chambers (25) and (27) meet in the mixing chamber, the syringe can be manipulated, for example, by shaking, to facilitate mixing to a single formulation which is injected by the syringe. Each chamber can also be configured to communicate directly with the needle if the substances in the two chambers do not necessarily need to be mix but it is still desired to administer them together.

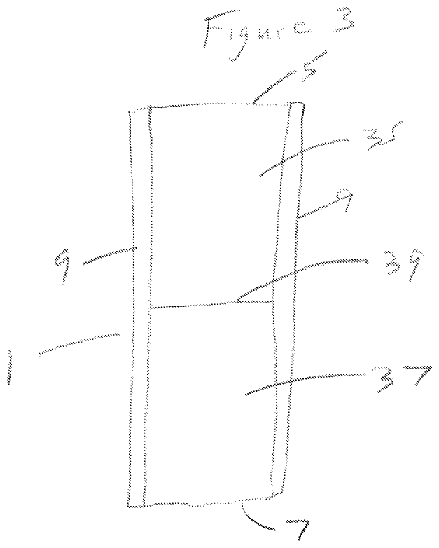

FIG. 3 illustrates a multi-chamber hollow tube section in which there are two chambers (35), (37) spaced linearly and sequentially along the hollow tubular section separated from one another by a breakable or breachable barrier (39). When the barrier is broken or breached, the contents of chamber (39) can pass into chamber (37), thereby mixing with the contents of chamber (38). When the contents of chambers (37) and (39) meet in chamber (37), the syringe can be manipulated to facilitate mixing to a single formulation which is injected by the syringe. This arrangement can be used to mix solids in chamber (37) with a diluent or solvent from chamber (39) to dissolve the solids for administration as a liquid or gel through the needle. This configuration is also well adapted for drugs that are prepared in lyophilized form and must be reconstituted with a diluent or solvent before using. The chambers can comprise the hollow tubular section itself or can be contained within an outer hollow tubular section.

The mixing chamber or any chamber in a multi-chambered hollow tubular section may be in a collapsed or flattened state before receiving the contents of another chamber and then expand outwardly or radially as it is filled with the contents of the other chamber. This is particularly useful when the proximal chamber contains a liquid that passes to a further distal chamber that contains solid or lyophilized contents and the liquid of the proximal chamber is passed to the more distal chamber and then the contents of both are mixed before injection into tissue.

Breakable or breachable barriers can be adapted to be broken, opened, or breached by the action of the syringe itself or by external application of pressure on the chambers, the barriers, or both, such as with the slideable collar described below. An example of a breakable or breachable barrier would be a thin section of the plastic used to blow mold the syringe or a light heat seal of the plastic used.

Alternatively, when the chambers are within an outer hollow tubular member, the chambers can be ruptured under direct or indirect pressure from the slideable collar or from an external force.

Referring to FIG. 1 again, the distal end of the hollow tubular section is adapted to receive and secure a needle containing piece (11). The needle containing piece includes a hollow needle (17) that has a tube piercing end (13) and an opposite discharging end (16). The needle may be of any suitable gauge for transporting the liquid, gas, or gel through it and is of a length suitable to pierce the distal end of the hollow tubular section and to deliver the formulation to targeted tissue of a subject in need of the liquid, gas, or gel. The needle may also be of different or varying gauges at any points or along its length. For example, the piercing end may be of wider gauge than the discharging end. The needle may also be of any suitable length to inject the formulation into a desire location such as, for example, subcutaneous, intramuscular, or intravenous. The needle containing piece also includes a retainer (49) that secures the needle containing piece to the syringe.

The distal end of the hollow tubular section is pierceable by the hollow tube piercing end of the needle to provide for open communication from the inside of said hollow tubular section for transport of any contents of the hollow tubular section thorough the needle discharge orifice. The needle piercing end may pierce the hollow tubular section to a depth sufficient for emptying the contents of the tube through the discharge end when the slideable collar is moved along the length of the tube.

Alternatively, the needle containing piece need not include a hollow tube piercing end. Rather, at least a portion of the distal end of the hollow tube itself, preferably the most distal portion, may be openable, such as by fracture, rupture, a valve, or a combination of any of the foregoing. Opening of this distal end portion will allow for open communication between the inside of the hollow tubular section and the needle, thereby allowing for discharge of the contents of the hollow tube portion through the needle containing piece discharge end. The distal end portion can be opened by pressure generated by the slideable collar's movement longitudinally along the hollow tubular section, preferably, the initial portion of the movement. Pressure will be generated on the contents of the hollow tubular section by the slideable collar. The contents will transfer this pressure to the distal end portion. This pressure will open the distal end portion. When a valve is used, the valve can be simply an openable valve or a valve that is openable under the pressure but closeable when the pressure is relieved such as by reverse or discontinued movement of the slideable collar. The distal end does not typically open until the user applies pressure through the slideable collar, thus, maintaining the aseptic nature of the syringe until use. The needle containing piece can be applied to the syringe during fill/finish or can be applied by the user before use. Opening can be facilitated by making the opening portion of the distal end thinner than the remainder of the hollow tubular section so that it ruptures under pressure from the slideable collar while the rest of the hollow tubular section does not. Alternatively, the portion of the distal end can be scored to make it fracturable along the score lines under pressure from the slideable collar while the rest of the hollow tubular section does not. Alternatively, the distal end portion can be thinner and scored. When the needle containing piece is supplied attached to the syringe, the entire device can be aseptically packaged, such as, for example, in a sleeve. Strips of syringes with needle containing pieces attached can be similarly packaged in a single sleeve which can be conformed to form individual sleeves for each syringe which can be separated from one another by the user, such as, for example, by tearing along perforated lines with each individual syringe maintaining its individual aseptic packaging until opened.

The syringe body also includes an extension piece (19) extending longitudinally from the proximal end of the hollow tubular section in a longitudinal direction away from the hollow tubular section. The extension piece can be flat, tubular, or of any three-dimensional configuration suitable for the syringe. The configuration of the extension piece may vary along its length. This extension piece provides a guide for the collar. It can also be of such thickness as to provide for extra rigidity of the syringe. The extension piece thickness and rigidity may vary along its length. The extension piece should not be of a shape, size, or length that impedes the movement of the slideable collar.

Preferably, the syringe body, and particularly, the hollow tubular section (particularly in the longitudinal direction, although most preferably in the radial direction also), is of sufficient rigidity (particularly longitudinal rigidity) to provide for placement of the needle containing piece, piercing of the distal end, insertion of the needle into tissue, and injection of any contents of the hollow tubular section, as well as breaking or breaching of any barriers between sections of a multi-chambered hollow tubular section. The syringe having a multi-chambered tubular section may also include a mixing chamber into which the contents of two or more chambers enters to be mixed by movement of the slideable collar before passing through a needle to tissue receiving the injection.

The syringe also includes a slideable collar (51). The slideable collar, before use of the syringe, is preferably located proximate or adjacent to the proximal end of the hollow tubular section, external to the hollow tubular section, and on the extension piece. The collar includes a compression portion (23) that is adapted to slide longitudinally along at least a portion of the extension piece and along one of more of the longitudinal flanges and the outer surface of the hollow tubular section to flatten or inwardly compress the hollow tubular section so that when a needle of a needle containing piece is piercing the distal end of the hollow tubular section, at least a portion of any contents of the hollow tubular section passes through the needle. The compression portion may be of any suitable shape to flatten or inwardly compress the hollow tubular section. For example, the surface of the compression portion that contacts the hollow tubular section may be flat. Alternatively, for example, the compressible portion may be comprised of one or more rollers, such as, for example a front and a back roller that roll along opposing sides of the hollow tubular section to flatten or inwardly compress the hollow tubular section. Alternatively, for example, the compressible portion may be a combination of a roller on one side and a flat section on the opposite side. Alternatively, for example, the compression portion may be flat on one side and a roller on the opposite side. The slideable collar, before use, can be attached to the syringe through a breakable joint that is adapted to break under direct or indirect pressure from the administrator so that it becomes free to slide along the hollow tubular section. This joint can be, for example, a perforation. The collar may be a closed collar which, when it slides along the hollow tubular section, completely surrounds that section, or it may be an open collar which only surrounds a portion of the tubular section sufficiently to substantially empty or completely empty the components of the hollow tubular section through the needle.

The extension piece can include a finger holder (21) for use by the administrator of the injection from the syringe. This finger holder can secure a finger of the administrator, in, or on and in the extension piece. The finger holder is preferably located between the proximal end of the extension piece and the proximal end of the tubular section and will assist the administrator in holding the syringe steady, inserting the needle into tissue, and applying pressure to move the slideable collar. The finger holder should not be of a shape, size, or length that impedes the movement of the slideable collar. For example, the finger holder can be a circular cut out of a size to accommodate the administrator's finger. The finger holder can be flap in the extension piece that is rotated into an open position to be used. When in the open position, the finger holder hole is open for finger insertion. When a finger is inserted into the finger holder, it assists the administrator in applying downward pressure on the slideable collar extension arms to move the slideable collar along the length of the filled hollow tubular section.

The slideable collar may include one or more arms (41) extending in the longitudinal direction of the extension piece and away from the hollow tubular section. Preferably, there are two extension arms, with preferably, one extending above each flange of the hollow tubular section. The arms can be split or grooved longitudinally so they can be guided to slide along the hollow tubular section flanges. The arms may be joined by a bridge (43) through which pressure can be applied to the arms to move the slideable collar along the outside of the hollow tubular section. The bridge may be of any shape that is comfortable for the application of finger pressure by the administrator, such as, for example, a disk.

The arms (41) of the slideable collar should be long enough so that the slideable collar (23) compression portion (23), when on extension (19) but before flattening the hollow tubular section, is below finger holder. The arms should also be long enough so that when the slideable collar compression portion (23) is at the distal end of the tube, the bridge (43) does not reach or cover the finger holder. This allows the slideable collar compression portion to travel the entire length of the hollow tube from the tubes proximal end to distal end without interference from the finger of the administrator that is contacting the finger holder.

The distal end of the hollow tubular section may include a mechanism for receiving and securing a needle containing piece, such as for example, a detent, an annular bulge (45) that fits into a receiving groove (47) in a needle containing piece (49), a groove that receives an annular bulge in the needle piece (49), screw threads to receive complimenting screw threads in a needle containing piece (49), a twist fit connection, friction fit, or the like.

Preferably, the syringes of the present invention are of one piece construction with the slideable collar being movable and essentially making the syringe two-piece when a force is applied to move the slideable collar. However, the syringes of the present invention may be made of more than one piece and assembled from such pieces. For example, the slideable collar may be a separate piece from the syringe body and could be placed on the syringe body by the manufacturer or user. The manufacturer or user would slip the slideable collar over the extension piece which then can move the slideable collar along the longitudinal direction of the extension piece onto the hollow tubular section, where the slideable collar can be moved longitudinally along the hollow tubular section.

The slideable collar assembly (which does not need to be aseptically manufactured or packaged since it never contacts the drug formulation or the patient) does not require a seal as does a traditional syringe plunger. It can also be manufactured and packaged separately from the syringe body. Furthermore, the slideable collar can be reusable.

The slideable collar can have grooves to accept ridges on the flanges to guide the slideable collar along the syringe.

The syringe may be comprised of plastic such as polypropylene (“PP”), high-density polyethylene (“HDPE”), low-density polyethylene (“LDPE”), ethylene vinyl alcohol (“EVOH”), or any combination of any of the foregoing. The plastic can be chosen by testing it by know methods for compatibility with the formulation to be injected.

The hollow tubular section of syringe of the present invention can be of various formulation volumes, such as volumes ranging from about 0.1 mL to about 500 mL. Preferred volumes range from about 1 mL to about 5 mL.

The syringe of the present invention is simple to use, is inexpensive to manufacture, is fast to manufacture and fill in any quantities, is aseptically manufactured with BFS, can be provided in handy strips, does not use air to move drug formulation through and out of the syringe which adds to the safety of the syringe, provides a similar administrator and patient experience as present day syringes, and is single-use for safety.

The syringe of the present invention can be blow molded such as, for example in a BFS process. Preferably, they are blow molded in one piece. BFS has become increasingly prevalent in the pharmaceutical industry and is now widely considered to be the superior form of aseptic processing by various medicine regulatory agencies including the U.S. Food and Drug Administration. BFS eliminates human intervention in aseptic packaging, making it a more robust method for the aseptic preparation of sterile pharmaceuticals.

Broadly, in BFS, a container is formed from pharmaceutical grade plastic resin, is filled with the drug formulation, and is sealed in a continuous process in a sterile enclosed area inside a machine. Specifically, the process is multi-stepped: first, pharmaceutical-grade plastic resin is vertically heat extruded through a circular throat to form a hanging tube. This extruded tube is then enclosed within a two-part mold, and the tube is cut above the mold. The mold is transferred to the filling zone, or sterile filling space, where filling needles or mandrels are lowered and used to inflate the plastic to form the container within the mold. Following the formation of the container, the needle or mandrel is used to fill the container with liquid. Following filling, the needles or mandrels are retracted, and a secondary top mold seals the container. All actions take place inside a sterile shrouded chamber inside the machine. The product is then discharged to a non-sterile area for labeling, packaging, and distribution. This is in contrast to traditional packaging where manufacturers had to follow a three-step process for packaging, manufacturing, and filling and sealing. Packaging formats were molded first and then transferred to the filling line where the packaging formats are filled and sealed. BFS is fast and precise. Moreover, automated BFS machines lead to low energy consumption, reduced waste generation, and lower carbon footprint. The BFS process can be adapted to fill more than one chamber of a syringe with sealing of a filled chamber before or concurrently with filling of another chamber. BFS can also provide a mixing chamber from which air is excluded and into which contents from multiple chambers are combined.

The substantial or total absence of air or other gases in any chambers of the hollow tubular section will assure that air or any other gas is not injected from the syringe into tissue.

Accordingly, the present invention provides a blow mold for the syringes of the invention and for a machine that uses these molds.

The syringe may be packaged with or without the needle containing piece and with or without the slideable collar.

The present invention also provides methods of injecting a liquid or gel formulation into an animal in need of such formulation.

Preferred formulations to be carried in the present syringes may include pharmaceuticals, biologics, vaccines, drugs, reagents, test components, or the like.

The needle containing piece is not necessary when, for example, the syringe is being used for a purpose other than injection of a formulation into an animal. For example, when the syringe is used to deliver a reagent such as in a test kit, something else may be used to pierce the distal end of the hollow tubular section so that the formulation in the chamber of that section can pass out of the syringe. For example, there can be provided a needle that is not part of an attachable needle containing piece to piece the distal end. Alternatively, the mold can also include a section for molding a plastic piece that can be used to pierce the distal end of the hollow tubular section. That plastic piece can be molded as part of the one piece molding of the syringe so that it is capable of being broken from the syringe, such as by a perforated joint, or so that it is attached through a flexible connector that is long enough so that the plastic piece can be used to pierce the distal end of the hollow tubular section without being removed from the syringe. The plastic piece can also be part of a cap that is connected to the syringe through a living hinge. The cap could be folded on the living hinge so that the piercing piece pierces the distal end of the hollow tubular section.

The animal can be any animal or species, preferably, a mammal, and most preferably, a human.

The syringes of the present invention can be single use syringes which assists in preventing abuse.

The syringes of the present invention may be imprinted and serialized.

The syringes of the present invention can be manufactured to contain any predetermined amount of drug formulation.

The syringes of the present invention can be manufactured in strips just as many BFS containers are presently produced. The slideable collar can be attached to the strip or can be a separate piece that the administrator simply slides onto the syringe body prior to use.

The syringes of the present invention dispense with the traditional internal plunger within a barrel. However, the administrator experience is similar to that with a traditional syringe. The drug formulation never contacts the environment during manufacturing, storage, or use.

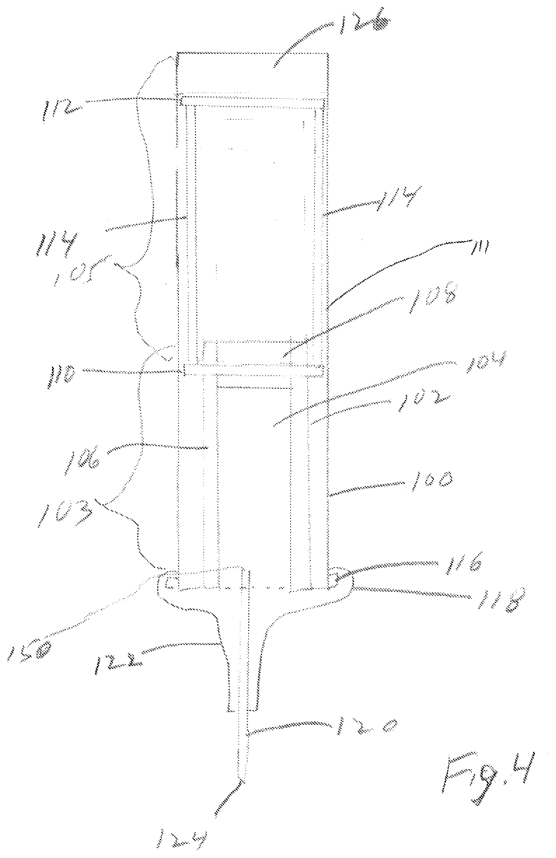

Referring to FIG. 4, the injector pen of the present invention includes an outer body (100) which is in a shape that is comfortable for the user to hold and manipulate such as, for example, a cylinder or a cylinder contoured with finger grooves for gripping the cylinder. The body can be of any suitable material such as, for example, plastic, metal, or a combination thereof. The body includes a lower area (103) for receiving a cartridge (102). This area is accessible for the user to insert a cartridge such as, for example, through a door, slide opening, port, or slit.

The body further includes an upper area (105) that accommodates a slideable collar (111). The slideable collar includes a compression portion (110) that is adapted to slide longitudinally along at least a portion of the cartridge (104) and along one or more of the longitudinal flanges (106) and the outer surface of the cartridge. The collar may be a closed collar which, when it slides along the cartridge, completely encircles the cartridge, or it may be an open collar which only encircles a portion of the tubular section sufficiently to substantially empty or completely empty the components of the cartridge through the needle. The slideable collar will flatten the cartridge when the collar passes over the cartridge. The compression portion may be of any suitable shape to flatten or inwardly compress the cartridge. For example, the surface of the compression portion that contacts the cartridge may be flat. Alternatively, for example, the compressible portion may be comprised of one or more rollers, such as, for example a front and a back roller that roll along opposing sides of the cartridge to flatten or inwardly compress the cartridge. Alternatively, for example, the compressible portion may be a combination of a roller on one side and a flat section on the opposite side. Alternatively, for example, the compression portion may be flat on one side and a roller on the opposite side.

The slideable collar is attached to one or more arms (114) extending opposite and away from the lower area of the device. The arms can be split or grooved longitudinally so they can be guided to slide along the hollow tubular section flanges. The arms may be joined by a bridge (112) through which pressure can be applied to the arms to move the slideable collar along the outside of the cartridge. The bridge may be of any shape that is suitable for transferring pressure to move the slideable collar such as, for example, a disk that fits within the upper area of the body but can travel in the longitudinal direction of the device.

The arms (114) of the slideable collar should be long enough so that the slideable collar compression portion (110) can travel the length of the cartridge sufficiently to empty a therapeutically effective amount of the formulation in the cartridge from the cartridge and through the needle (120).

The slideable collar extension arms, and bridge can be shaped to match the inner contour of the device body.

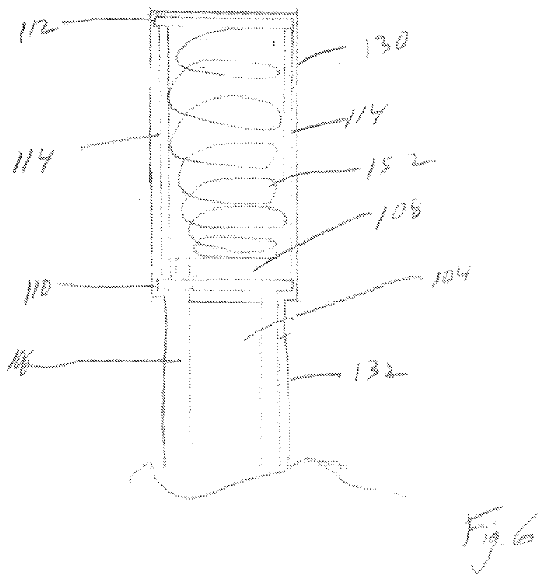

The slideable collar (through its arms) is moved by a driver located at or in the upper end (126) of the device. The driver preferably is user activated. The driver may be, for example, a motor, a spring, a preloaded spring, a ratchet, or the like. Alternatively, for example, a portion of the bridge can project outside of the outer wall of the device and include areas to which finger pressure can be applied to drive the slideable collar over the cartridge. Alternatively, for example, the body can be of a cap and sleeve construction whereby the cap portion slides over the sleeve portion to move the slideable collar downwardly. There is a release for the driver that is user accessible. There can also be provided a mechanism to raise the slideable collar to its initial position after it has slide along the cartridge, such as, for example, a return spring (156), as shown in FIG. 6.

Referring to FIG. 4, the cartridge includes a hollow compressible tubular section (104) and an extension (108). The hollow tubular section is flattenable or compressible inwardly (preferably in a radial direction) along a majority of its longitudinal length to flatten or compress at least a majority of the tube while maintaining the longitudinal length of the tube and, preferably, along its longitudinal length sufficient to empty substantially all (e.g., a therapeutically effective amount of the dose) of any liquid or gel contents from the hollow tubular section. Preferably, once the hollow tubular section has been flattened, it need not or does not substantially rebound to a non-flattened or compressed state. The hollow tubular section need not be compressible in the longitudinal direction in any amount sufficient to assist in evacuating liquid or gel from the hollow tubular section. This tubular section, similar to the prefilled syringe described above, has a closed proximal end, a closed distal end, and at least one longitudinal flange (106) extending along at least a majority of the length of the tubular section. Preferably, flange(s) extend longitudinally along the entire tubular section. The flange(s) may also include a guide ridge or rail or the like in the longitudinal direction along at least a portion of the flange that will accept and guide the slideable collar to pass over and flatten the tubular section. The proximal and distal ends of the tubular section are sealed in order to contain a liquid or gel in the hollow tubular section that will be injected by the syringe.

The hollow tubular section may be completely cylindrical or may be flat or another shape along a portion of its length or entirety. Preferably, the hollow tubular section will taper towards its distal end, it proximal end, or both, for example, as would be formed in heat sealing a plastic.

The hollow tubular section can form a single chamber or a plurality of chambers. The plurality of chambers can be arranged linearly one after the other (e.g., in tandem), parallel to one another, or in a combination thereof. There could be a breakable or breachable barrier or connection between the chambers. Preferably, the barrier or connection would be broken or breached by pressure directly or indirectly from the slideable collar (e.g., the slideable collar is moved along the tubular section sufficiently to force the contents of the upper chamber to break or breach the barrier or connection between the two chambers with the contents of the upper chamber then emptying into the lower chamber). A multi-chamber tubular section could allow for the administration of more than one liquid or gel in a single injection or the mixing of multiple components (at least one being a liquid or gel) in the syringe itself before or during injection. The length of travel of the slideable collar can be regulated to empty the contents of the upper chamber into the lower chamber and to pause to allow for mixing. Thereafter, the slideable collar could be lowered further to inject the mixture. This would be particularly suitable for lyophilized formulations that must be reconstituted with a diluent before administration, wherein the upper most chamber contains the diluent and the lower chamber contains the lyophilized product.

The lower end of the device is adapted to receive and secure a needle containing piece (122). The needle containing piece includes a hollow needle (120) that has a hollow tube piercing end (150) and an opposite discharging end (124). The needle may be of any suitable gauge for transporting the liquid, gas, or gel through it and is of a length suitable to pierce the distal end of the hollow tubular section and to deliver the formulation to targeted tissue of a subject in need of the liquid, gas, or gel. The needle may also be of different or varying gauges at any points or along its length. For example, the piercing end may be of wider gauge than the discharging end.

Alternatively, the needle containing piece need not include a hollow tube piercing end. Rather, at least a portion of the distal end of the hollow tube itself, preferably the most distal portion, may be openable, such as by fracture, rupture, a valve, or a combination of any of the foregoing. Opening of this distal end portion will allow for open communication between the inside of the hollow tubular section and the needle, thereby allowing for discharge of the contents of the hollow tube portion through the needle containing piece discharge end. The distal end portion can be opened by pressure generated by the slideable collar's movement longitudinally along the hollow tubular section, preferably, the initial portion of the movement. Pressure will be generated on the contents of the hollow tubular section by the slideable collar. The contents will transfer this pressure to the distal end portion. This pressure will open the distal end portion. When a valve is used, the valve can be simply an openable valve or a valve that is openable under the pressure but closeable when the pressure is relieved such as by reverse or discontinued movement of the slideable collar. The distal end does not typically open until the user applies pressure through the slideable collar, thus, maintaining the aseptic nature of the syringe until use. The needle containing piece can be applied to the syringe during fill/finish or can be applied by the user before use. Opening can be facilitated by making the opening portion of the distal end thinner than the remainder of the hollow tubular section so that it ruptures under pressure from the slideable collar while the rest of the hollow tubular section does not. Alternatively, the portion of the distal end can be scored to make it fracturable along the score lines under pressure from the slideable collar while the rest of the hollow tubular section does not. Alternatively, the distal end portion can be thinner and scored. When the needle containing piece is supplied attached to the syringe, the entire device can be aseptically packaged, such as, for example, in a sleeve. Strips of syringes with needle containing pieces attached can be similarly packaged in a single sleeve which can be conformed to form individual sleeves for each syringe which can be separated from one another by the user, such as, for example, by tearing along perforated lines with each individual syringe maintaining its individual aseptic packaging until opened.

The needle may also be of any suitable length to inject the formulation into a desire location such as, for example, subcutaneous, intramuscular, or intravenous. The needle piercing end may pierce the hollow tubular section to a depth sufficient for emptying the contents of the tube through the discharge end when the slideable collar is moved along the length of the cartridge. The needle containing piece also includes a retainer that secures the needle containing piece to the syringe.

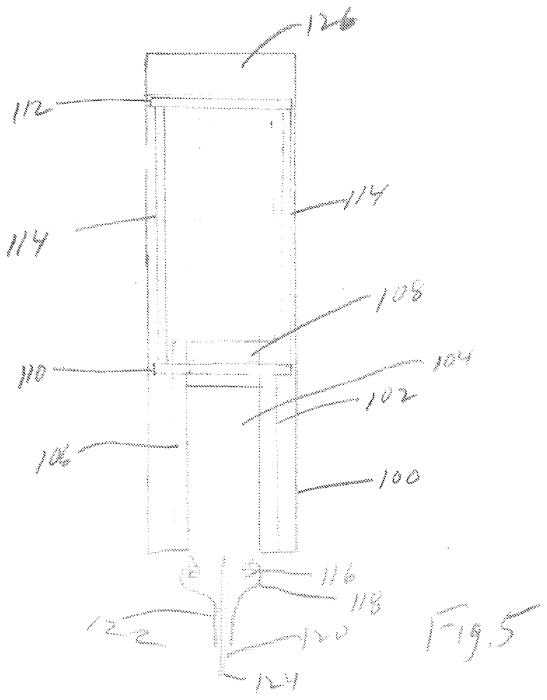

Referring to FIGS. 4 and 5, the lower end of the device or cartridge may include a mechanism for receiving and securing a needle containing piece, such as for example, a detent, an annular bulge (116) that fits into a receiving groove (118) in a needle containing piece (122), a groove that receives an annular bulge in the needle piece (122), screw threads to receive complimenting screw threads in a needle containing piece (122), a twist fit connection, friction fit, or the like.

Alternatively, for example, the cartridge itself can have a section adapted to receive and secure a needle containing piece. The device can be structured so that the distal end of the cartridge protrudes from the lower end of the device. Alternatively, for example, there can be a recess in the device to make the lower end of the cartridge accessible for needle attachment.

The lower end of the cartridge is pierceable by the hollow tube piercing end of the needle to provide for open communication from the inside of the cartridge for transport of any contents of the cartridge thorough the needle discharge orifice.

Preferably, the cartridge, and particularly, the hollow tubular section (particularly in the longitudinal direction), is of sufficient rigidity (particularly longitudinal rigidity) so that a full cartridge does not collapse, flatten, or compress longitudinally in the device in an amount that adversely affects or impedes injection of the cartridge contents or in an amount sufficient to eject more than an insignificant amount of the formulation from the cartridge before the entire dose of the cartridge is delivered to the tissue. An insignificant amount is an amount that does not adversely affect the therapeutic efficacy of the amount of the formulation in the full cartridge. Preferably, the cartridge is also of sufficient rigidity in the longitudinal direction to facilitate or accommodate insertion of a needle containing piece into the cartridge and into tissue. This can be achieved by the cartridge itself or by supporting structure within the device. These longitudinal rigidity conditions are applicable to a full cartridge but not necessarily applicable to a cartridge after its contents have been injected into a tissue.

The slideable collar assembly does not require a seal as does a traditional syringe plunger.

The slideable collar can have grooves to accept ridges on the flanges to guide the slideable collar along the cartridge.

The cartridge may be comprised of plastic such as polypropylene (“PP”), high-density polyethylene (“HDPE”), low-density polyethylene (“LDPE”), ethylene vinyl alcohol (“EVOH”), or any combination of any of the foregoing. The plastic can be chosen by testing it by know methods for compatibility with the formulation to be injected.

The cartridges of the present invention can be of various formulation volumes, such as volumes ranging from about 0.1 mL to about 500 mL. Preferred volumes range from about 1 mL to about 5 mL. The cartridges of the present invention can be manufactured to contain any predetermined amount of drug formulation.

The cartridges of the present invention may be imprinted and serialized.

The cartridges of the present invention can be manufactured in strips, and the injector pen can be structured to receive the first cartridge of a strip through a loading port or door without separating it from the remainder of the full cartridges in the strip, so that other cartridges in the strip can be sequentially moved into the device's lower end. In such an embodiment, the slideable collar could have an opening for receiving the cartridge extension laterally and the arm above such an opening could have a corresponding slit to allow the arm to move along the cartridge flange without the necessity of breaking off other cartridges in the strip. The empty cartridge would be removed from an exit port on the device and would be detached from the strip while the next cartridge of the strip enters to lower end of the device.

The discharge end of the needle could be enclosed in a sterile protective cover that would also be pierced or removed when the device is activated or that can be removed by the user before administration depending in the embodiment of the device. For example, if the needle discharging end is exposed outside of the device's body before activation, the cover could easily be removed by the user before activation. If the needle discharging end is not exposed outside of the device's body before activation, the cover could be pierced and collapsed during activation, preferably while the cartridge is being inserted into the needle containing device. However, the piercing of the cover could happen at any point after activation so long as the cover is pierced before the needle discharge end is inserted into the tissue. Similarly, the opposite end of the needle or the entire needle containing piece can be enclosed in a sterile protective cover that can be removed by the user or can be pierced by activation of the device. This can be the same as or different from the cover of the other end of the needle. Alternatively, the entire needle containing piece can be in a sterile cover that is either removed or pierced as above. Sterile covers can be made from, for example, plastic, paper, foil, or the like.

The injector pens of the present invention are reusable with a new cartridge and new needle for each use.

Referring again to FIG. 4, an unused (i.e., full) cartridge (102) is placed in the lower end (103) of the device so that at least a portion of the cartridge's extension (108) is inserted through the slideable collar (110). The needle piece would then be placed on the device or cartridge, thereby piercing the cartridge. Alternatively, the needle could be placed on the cartridge or device, and then, the cartridge could be inserted into the device. The user would place the needle into the tissue and then activate the driver which would move the slideable collar to travel along the length of the cartridge to empty the contents of the cartridge into the tissue.

Alternatively, the device could be adapted to receive through a door, port, or the like, a cartridge adapted to hold the needle containing piece and a needle containing piece in tandem. The driver would activate the slideable collar to drive the needle receiving end of the cartridge into the needle containing piece, thereby piercing the cartridge. Once the cartridge was pierced, the slideable collar would proceed to flatten the cartridge, thereby delivering the cartridge contents through the needle.

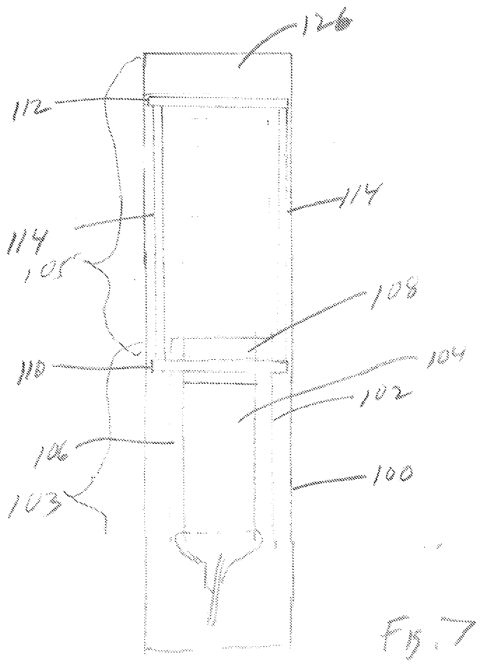

In a further embodiment, for example as shown in FIG. 7, the needle would not protrude from the device until the slideable collar was driven to have the needle pierce the cartridge and the slideable collar would proceed to drive the needle into the tissue by moving the cartridge longitudinally in the device sufficiently to place the needle in the tissue and then would empty the contents of the cartridge into the tissue. A return mechanism such, as, for example, a return spring would cause the needle to retreat into the device once the cartridge's contents are discharged and the slideable collar retreats. This embodiment is particularly well suited for emergency administration of drugs for anaphylaxis or drug overdose. The device could be reloadable also, rather than disposable.

Any of the injector pen bodies of the present invention may be made in two or more pieces. For example, the body may be divided into a top piece that includes the driver and the slideable collar and a bottom piece that accepts the needle containing piece and the cartridge. For example, when the needle containing piece and the cartridge are placed in the bottom piece of the body (which can dispense of another opening for inserting the cartridge or needle containing piece), a detent, or the like. The top piece and the slideable collar can be place over cartridge's extension and the top and bottom pieces can be mated to one another such as, for example, by friction, a lock, twisting, and the like. The injector pen can then be activated.

Structural guides can be part of the body (e.g., molded into the body) of any embodiment to assure that the needle containing piece and cartridge are properly inserted and are inserted in the correct direction.

The device can also contain the exposed needle containing device portion and the cartridge that is pierced by movement of the cartridge by the slideable collar, particularly in a linear arrangement with the needle in line for piercing the cartridge. For example, the needle containing device can be inserted into the lowest part of the body, followed by the cartridge, and then, the device would be closed, if the body were in two parts, the two parts would be connected. The piercing action can be manually performed, performed when the driver is activated, or a combination of both.

There can also be a mechanism that prevents accidental activation of the injector pen such as a safety or a lock out. The injector pen may also be password protected to prevent unauthorized use.

The device can be provided with disposable protective cover that prevents direct contact of the device with the patient or any bodily fluids from the patient. The cover would be disposed of after each administration. This would allow the injector pen to be used for multiple patients without sterilization or cleaning between patients. The device can also include a mechanism that provides oral instructions for use when the device is activated. The different ends of the device can be individually color coded so that the user places the device in the proper direction for injection.

The cartridges of the present invention are simple to use, are inexpensive to manufacture, are fast to manufacture and fill in any quantities, are aseptically manufactured with BFS.

The present invention provides a blow mold for the cartridges of the invention and for a machine that uses these molds.

The injector pen body can be manufactured by methods known in the art, such as, for example, molding, extrusion, or the like.

The present invention also provides methods of injecting a liquid or gel formulation into an animal in need of such formulation.

Preferred formulations to be carried in the present cartridges may include pharmaceuticals, medications, biologics, vaccines, drugs, reagents, test components, or the like.

The animal can be any animal or species, preferably, a mammal, and most preferably, a human.

Force from an external slideable collar directed to the outside of a hollow tubular section drives the injection of the contents in the syringes and injector pens of the present invention. It is not necessary for air or internal fluids that dilute the formulation to be injected for injection.

The syringes and injector pens of the present invention have many advantages over traditional glass-based fill/finish products such as, for example, robust unbreakable user-friendly containers; unit dose accessibility; improved patient adherence; no glass spalling (i.e., less than 10% of conventional glass particle contamination); no silicone; no need for cleaning/sterilization of already-manufactured containers; flexibility of container shapes and sizes; straightforward adaptation to specific applications and administration regimens; superiority for highly sensitive formulations; ISO Class 5 safety conditions; elimination of some or all preservatives; fully automated processing (i.e., little or no human intervention); higher overall speed; significantly smaller space requirements for supplies and processing equipment; high process and product reliability; lower manufacturing costs; lower manufacturing personnel requirements and resultant lower labor costs; and shorter and more reliable production and logistics chains with no requirements for separate sourcing, delivery, and handling of glass containers. They can also effectively tackles the counterfeit drug menace and are particularly advantageous for expensive drugs. Both provide a similar administrator and patient experience as present day syringes and injector pens, which means that they can be readily and willingly adopted by users without significant retraining.

The term “about” means within a range allowing for the error inherent in the measuring mechanism.

The term “for example” is non-limiting.

A “therapeutically effective amount” is that amount which is effective to prevent, treat, ameliorate, or cure a medical condition. This amount will depend upon the drug, biologic, and patient. Most drugs, biologics, and vaccines are labelled with its respective therapeutically effective amounts. If there is no such label, a therapeutically effective amount can be determined by those skilled in the art. Drugs, biologics, and vaccines useful in the present invention may also be formulated with excipients.

All patents, applications, citations, literature, and publications mentioned herein are hereby expressly incorporated by reference in their entirety.

Many variations of the present invention will suggest themselves to those skilled in this art in light of the above, detailed description. All such obvious variations are within the full scope of the appended claims.

Aspects of the invention include:

Aspect 1: A prefilled syringe comprising:
(a) a one piece body, the body comprising:
   (i) a hollow tubular section at least partially and preferably substantially completely non-longitudinally flattenable under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a hollow tube piercing end and a discharge end;
      the distal end being pierceable by the needle hollow tube piercing end of the needle to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue and wherein said hollow tubular section contains a therapeutically effective amount of a pharmaceutical or biological drug or vaccine; and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, preferably, compress radially, the hollow tubular section so that when a needle containing piece is piercing the distal end of the hollow tubular section, at least a portion of any contents of the hollow tubular section passes through the needle discharge end.

Aspect 2. A syringe as defined in aspect 1, wherein said syringe is made from a material selected form the group consisting of polypropylene (“PP”), high-density polyethylene (“HDPE”), low-density polyethylene (“LDPE”), ethylene vinyl alcohol (“EVOH”), or any combination of any of the foregoing.

Aspect 3. A syringe as defined in aspect 1, wherein said hollow tubular section has two opposing flanges extending along at least a majority of the length of said tubular section.

Aspect 4. A syringe as defined in aspect 1, wherein said hollow tubular section contains a therapeutically effective amount of a pharmaceutical or biological drug or vaccine.

Aspect 5. A device comprising a syringe as defined in aspect 1 and an attached needle containing piece.

Aspect 6. A kit comprising a syringe as defined in aspect 1 and a needle containing piece.

Aspect 7. A kit comprising a plurality of syringes as defined in aspect 1, wherein said syringes are joined together on at least a portion of their longitudinal sides as a strip.

Aspect 8. A blow fill seal machine adapted to manufacture a syringe as defined in aspect 1.

Aspect 9. A blow fill mold for preparing a syringe as defined in aspect 1.

Aspect 10. A blow fill mold adapted to manufacture a syringe as defined in aspect 1.

Aspect 11. A method of injecting a therapeutically effective amount of a pharmaceutical or biological drug or vaccine into an animal in need of said pharmaceutical or biological drug or vaccine, said method comprising:
(a) providing a syringe as defined in aspect 4;
(b) placing on said syringe, a needle containing piece, said needle containing piece comprising a retainer and a needle, said needle having a hollow tube piercing end and a discharge end, whereby said hollow tube piercing end of said needle pierces the distal end of the hollow tubular section;
(c) inserting the needle discharge end of said needle through at least a portion of one or more tissues of said animal; and
(d) sliding said slideable collar longitudinally along at least a portion of the outside of said hollow tubular section, thereby inwardly compressing at least a portion of said hollow tubular section to force a therapeutically effective amount of a pharmaceutical or biological drug or vaccine through said needle discharge end and into said tissue.

Aspect 12. A syringe comprising:
(a) a one piece body, the body comprising:
   (i) a hollow tubular section at least partially and preferably substantially completely non-longitudinally flattenable under direct or indirect force from a slideable collar, preferably, at least partially radially collapsible or compressible, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a a discharge end;
      at least a portion of the distal end being permanently openable when a slideable collar is moved longitudinally along said hollow tubular section to provide for open communication from the inside of the hollow tubular section for transport of any contents of the hollow tubular section through the needle discharge end; wherein said hollow tubular section cannot collapse longitudinally in an amount that adversely impedes injection of syringe contents in an amount sufficient to eject more than an insignificant amount of the formulation from the syringe before the entire dose of the syringe is delivered to tissue; and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten the hollow tubular section so that when a needle containing piece is attached the distal end of the hollow tubular section and said at least a portion of said distal end of said hollow tubular section is open, at least a portion of any contents of the hollow tubular section passes through the needle discharge end.

Aspect 13. A prefilled syringe comprising:
(a) a one piece body, the body comprising:
   (i) a hollow tubular section at least partially and preferably substantially completely non-longitudinally flattenable under direct or indirect force from a slideable collar, the hollow tubular section having a closed proximal end, a closed distal end, and at least one flange extending along at least a majority of the length of the tubular section;
      the distal end adapted to receive and secure a needle containing piece, the needle containing piece comprising a retainer and a needle, the needle having a a discharge end;
      at least a portion of the distal end being permanently openable, or openable and closeable, preferably by fracture, rupture, or valve, when a slideable collar is moved longitudinally along said hollow tubular section, preferably by pressure generated by the movement of said slideable collar on any contents of said hollow tubular section, to provide for open communication from the inside of the hollow tubular section for transport of any contents of the hollow tubular section through the needle discharge end;
      wherein said hollow tubular section cannot collapse, flatten, or compress longitudinally in an amount sufficient to eject more than an insignificant amount of the formulation from the syringe before the entire dose of the syringe is delivered to tissue and wherein said hollow tubular section contains a therapeutically effective amount of a pharmaceutical or biological drug or vaccine; and
   (ii) an extension piece extending longitudinally from the proximal end of the hollow tubular section and in a direction opposite and away from the hollow tubular section; and
(b) a slideable collar on the extension piece, proximate or adjacent to the proximal end of the hollow compressible tubular section, and external to the tubular section, the collar comprising a compression portion, the compression portion being adapted to slide longitudinally along the longitudinal flanges and the outer surface of the hollow tubular section to flatten, the hollow tubular section so that when a needle containing piece is attached the distal end of the hollow tubular section and said at least a portion of said distal end of said hollow tubular section is open, at least a portion of any contents of the hollow tubular section passes through the needle discharge end.

Aspect 14. A method of injecting a liquid or gel into an animal in need of the liquid or gel with the syringe described above, the method comprising:
(a) providing a syringe as defined on Aspect 11 with the liquid or gel in the hollow tubular section;
(b) placing on the syringe, a needle containing piece, the needle containing piece comprising a retainer and a needle;
(c) inserting the needle discharge end of the needle through at least a portion of one or more tissues of the animal; and
(d) sliding the slideable collar longitudinally along at least a portion of the outside of the hollow tubular section, thereby opening at least a portion of the distal end of the hollow tubular section and compressing at least a portion of the hollow tubular section to force at least a portion of the liquid or gel through the needle discharge end and into the tissue.

Aspect 15. An injector pen comprising:
(a) an outer body having a lower cartridge receiving area, an upper slideable collar mechanism area, and a needle containing piece receiver;
(b) a cartridge in said lower cartridge receiving area, said cartridge comprising (i) a hollow tubular section at least partially and preferably substantially completely non-longitudinally flattenable (i.e., inwardly collapsible or compressible) under direct or indirect force from a slideable collar, preferably, at least partially radially collapsible or compressible, said hollow tubular section having a closed proximal end, a closed distal end, and at least one flange extending along at least a majority of the length of said hollow tubular section and said distal end being pierceable by a needle of a needle containing piece to provide for open communication from the inside of said cartridge for transport of any contents of said hollow tubular section through a needle the needle discharge end of said needle containing piece; wherein said hollow tubular section does not collapse, flatten, or compress longitudinally in an amount that adversely impedes injection of the cartridge contents in an amount sufficient to eject more than an insignificant amount of the formulation from the cartridge before the entire dose of the cartridge is delivered to tissue; (ii) an extension piece extending longitudinally from said proximal end of said hollow tubular section and in a direction opposite said hollow tubular section; and (iii) a therapeutically effective amount of a pharmaceutical or biological drug or vaccine contained in said hollow tubular section;
(c) a slideable collar in said upper area, said slideable collar comprising a compression portion, said compression portion being adapted to slide longitudinally along longitudinal flanges and outer surface of said cartridge to flatten said cartridge, one or more extension arms extending in a longitudinal direction of said injector pen away from said cartridge receiving area opposite from said compression piece; a bridge connecting said extension arms at their respective ends opposite said compression portion, and a user activated slideable collar driver; and
(d) a needle on said needle containing piece, said needle containing piece comprising a retainer and a needle, said needle having a hollow tube piercing end and a discharge end.

Aspect 16. A method of injecting a therapeutically effective amount of a pharmaceutical or biological drug or vaccine into an animal in need of said pharmaceutical or biological drug or vaccine, said method comprising:
(a) providing an injector pen as defined in Aspect 13;
(b) placing said needle discharge end into a tissue of said animal; and
(c) activating said slideable collar to move longitudinally along at least a portion of the outside of said cartridge, thereby flattening at least a portion of said hollow tubular section of said cartridge to force at least a portion of said liquid or gel through said needle discharge end and into said tissue.

Aspect 17. An injector pen comprising:
(a) an outer body having a lower cartridge receiving area, an upper slideable collar mechanism area, and a needle containing piece receiving area below said cartridge receiving area;
(b) a cartridge in said lower cartridge receiving area, said cartridge comprising (i) a hollow tubular section at least partially and preferably substantially completely non-longitudinally flattenable (i.e., inwardly collapsible or compressible) under direct or indirect force from a slideable collar, preferably, at least partially radially collapsible or compressible, said hollow tubular section having a closed proximal end, a closed distal end, and at least one flange extending along at least a majority of the length of said hollow tubular section and said distal end being pierceable by a needle of a needle containing piece to provide for open communication from the inside of said cartridge for transport of any contents of said hollow tubular section through a needle the needle discharge end of said needle containing piece; wherein said hollow tubular section does not collapse, flatten, or compress longitudinally in an amount that adversely impedes injection of the cartridge contents in an amount sufficient to eject more than an insignificant amount of the formulation from the cartridge before the entire dose of the cartridge is delivered to tissue; (ii) a needle receiving section at said distal end, (iii) an extension piece extending longitudinally from said proximal end of said hollow tubular section and in a direction opposite said hollow tubular section, and (iv) a therapeutically effective amount of a pharmaceutical or biological drug or vaccine;
(c) a slideable collar in said upper area, said slideable collar comprising a compression portion, said compression portion being adapted to slide longitudinally along longitudinal flanges and outer surface of said cartridge to flatten said cartridge, one or more extension arms extending in a longitudinal direction of said injector pen away from said cartridge receiving area opposite from said compression piece; a bridge connecting said extension arms at their respective ends opposite said compression portion, and a user activated slideable collar driver, wherein at least a portion of said extension piece of said cartridge is inserted into said slideable collar; and
(d) a needle containing piece in said needle piece receiving, said needle containing piece being proximate to said needle receiving section of said cartridge, said needle containing piece comprising a needle having a hollow tube piercing end and a discharge end;
whereby when said slideable collar moves longitudinally toward said tubular section of said cartridge, said cartridge needle receiving section engages said needle containing piece and said hollow tube piercing section pierces said distal end of said cartridge thereby opening communication between any contents of said cartridge and said needle, whereby when said slideable collar moves further longitudinally toward said tubular section of said cartridge said needle protrudes from said device in an amount sufficient to penetrate tissue of an animal in need of such formulation, and when slideable collar moves longitudinally over said hollow tubular section, said therapeutically effective amount of a pharmaceutical or biological drug or vaccine is injected into said tissue.

Aspect 18. An injector pen as defined in Aspect 17, further comprising a needle retractor adapted to retract said needle into said body after the formulation has been injected into said tissue.

Aspect 19. An injector pen as defined in Aspect 17, wherein said cartridge is made from a material selected form the group consisting of polypropylene (“PP”), high-density polyethylene (“HDPE”), low-density polyethylene (“LDPE”), ethylene vinyl alcohol (“EVOH”), or any combination of any of the foregoing.

Aspect 20. An injector pen as defined in aspect 17, wherein said hollow tubular section has two opposing flanges extending along at least a majority of the length of said tubular section.

Aspect 21. A blow fill seal machine adapted to manufacture an injector pen cartridge as defined in aspect 17.

Aspect 22. A blow fill mold for preparing an injector pen cartridge as defined in aspect 17.

Aspect 23. A blow fill mold adapted to manufacture an injector pen cartridge as defined in aspect 17.

Aspect 24. A method of injecting a liquid or gel into an animal in need of said liquid or gel, said method comprising:
(a) providing an injector pen as defined in aspect 17;
(b) placing said injector pen against said animal; and
(c) actuating said injector pen to move said slideable collar longitudinally toward said hollow tubular section of said cartridge, said cartridge needle receiving section engaging said needle containing piece and said hollow tube piercing section piercing said distal end of said cartridge thereby opening communication between any contents of said cartridge and said needle, further to move said slideable collar further longitudinally toward said tubular section of said cartridge to move said needle to protrude from said device in an amount sufficient to penetrate tissue of an animal in need of such formulation, and further to move said slideable collar moves longitudinally over said hollow tubular section to inject said therapeutically effective amount of a pharmaceutical or biological drug or vaccine into said tissue from said needle discharging end.

Aspect 25. An injector pen comprising:
(a) an outer body having a lower cartridge receiving area, an upper slideable collar mechanism area, and a needle containing piece receiver;
(b) a cartridge in the lower cartridge receiving area, the cartridge comprising:
   (i) a hollow tubular section at least partially non-longitudinally flattenable, the hollow tubular section having a closed proximal end, a closed distal end, and at least one flange extending along at least a majority of the length of the hollow tubular section and the distal end adapted to receive and secure a needle containing piece, the needle containing piece comprising a retainer and a needle, the needle having a discharge end;
      at least a portion of the distal end being permanently openable when a slideable collar is moved longitudinally along said hollow tubular section to provide for open communication from the inside of the hollow tubular section for transport of any contents of the hollow tubular section through the needle discharge end;
   (ii) an extension piece extending longitudinally from the proximal end of said hollow tubular section and in a direction opposite and away from the hollow tubular section; wherein said hollow tubular section does not collapse longitudinally in an amount that adversely impedes injection of cartridge contents in an amount sufficient to eject more than an insignificant amount of the formulation from said cartridge before the entire dose of the cartridge delivered to tissue; and
   (iii) a therapeutically effective amount of a pharmaceutical or biological drug or vaccine;
(c) a slideable collar in the upper area, the slideable collar comprising a compression portion, the compression portion being adapted to slide longitudinally along longitudinal flanges and outer surface of the cartridge to flatten the cartridge, one or more extension arms extending from the compression piece in a longitudinal direction of the injector pen opposite and away from the cartridge receiving area opposite from the compression piece; a bridge connecting the extension arms at their respective ends opposite the compression portion, and a user activated slideable collar driver; and
(d) a needle containing piece on the needle containing piece receiver, said needle containing piece comprising a retainer and a needle, the needle having a discharge end.

Aspect 26. A method of injecting a liquid or gel drug formulation into an animal in need of said liquid or gel, said method comprising:
(a) providing an injector pen as defined in aspect 25;
(b) placing said needle discharge end into a tissue of said animal; and
(c) activating said slideable collar to move longitudinally along at least a portion of the outside of said cartridge, thereby opening at least a portion of the distal end of the hollow tubular section and flattening at least a portion of said hollow tubular section of said cartridge to force said therapeutically effective amount of said drug formulation through said needle discharge end and into said tissue.

Aspect 27. An injector pen comprising:
(a) an outer body having a lower cartridge receiving area, an upper slideable collar mechanism area, and a needle containing piece receiving area below said cartridge receiving area;
(b) a cartridge in said lower cartridge receiving area, said cartridge comprising:
   (i) a hollow tubular section at least partially non-longitudinally flattenable under direct or indirect force from a slideable collar, said hollow tubular section having a closed proximal end, a closed distal end, and at least one flange extending along at least a majority of the length of said hollow tubular section and said distal end adapted to receive and secure a needle containing piece, the needle containing piece comprising a retainer and a needle, the needle having a discharge end;
      at least a portion of the distal end being permanently openable when a slideable collar is moved longitudinally along said hollow tubular section to provide for open communication from the inside of the hollow tubular section for transport of any contents of the hollow tubular section through the needle discharge end; wherein said hollow tubular section does not collapse longitudinally in an amount that adversely impedes injection of the syringe contents in an amount sufficient to eject more than an insignificant amount of the formulation from the syringe before the entire dose of the syringe is delivered to tissue;
   (ii) a needle receiving section at said distal end,
   (iii) an extension piece extending longitudinally from said proximal end of said hollow tubular section and in a direction opposite said hollow tubular section, and
   (iv) a therapeutically effective amount of a pharmaceutical or biological drug or vaccine contained in said hollow tubular section;
(c) a slideable collar in said upper area, said slideable collar comprising a compression portion, said compression portion being adapted to slide longitudinally along longitudinal flanges and outer surface of said cartridge to flatten said cartridge, one or more extension arms extending in a longitudinal direction of said injector pen away from said cartridge receiving area opposite from said compression piece; a bridge connecting said extension arms at their respective ends opposite said compression portion, and a user activated slideable collar driver, wherein at least a portion of said extension piece of said cartridge is inserted into said slideable collar; and
(d) a needle containing piece in said needle piece receiving, said needle containing piece being proximate to said needle receiving section of said cartridge, said needle containing piece comprising a discharge end;
whereby when said slideable collar moves longitudinally toward said tubular section of said cartridge, said cartridge needle receiving section engages said needle containing piece and said at least a portion of the distal end opens thereby opening communication between any contents of said cartridge and said needle, whereby when said slideable collar moves further longitudinally toward said tubular section of said cartridge said needle protrudes from said device in an amount sufficient to penetrate tissue of an animal in need of such formulation, and when slideable collar moves longitudinally over said hollow tubular section, said therapeutically effective amount of a pharmaceutical or biological drug or vaccine is injected into said tissue.

Aspect 28. A method of injecting a liquid or gel into an animal in need of said liquid or gel, the method comprising:
(a) providing an injector pen as defined in aspect 27;
(b) placing said injector pen against said animal; and
(d) actuating said injector pen to move slideable collar longitudinally toward said tubular section of said cartridge, said cartridge needle receiving section engaging said needle containing piece and at least a portion of said distal end of said hollow tube opening, thereby opening communication between any contents of said cartridge and said needle, further to move said slideable collar further longitudinally toward said tubular section of said cartridge to move said needle to protrude from said device in an amount sufficient to penetrate tissue of an animal in need of such formulation, and further to move said slideable collar longitudinally over said hollow tubular section to inject said therapeutically effective amount of a pharmaceutical or biological drug or vaccine into said tissue from said needle discharging.

1. A syringe comprising:
(a) a one piece body, the body comprising:
(i) an inwardly compressible hollow tubular section, said hollow tubular section having a closed proximal end, a closed distal end, and at least one flange extending along at least a majority of the length of said hollow tubular section, wherein said hollow tubular section is at least partially non-longitudinally flattenable (under direct or indirect force from a slideable collar);
said distal end adapted to receive and secure a needle containing piece, said needle containing piece comprising a retainer and a needle, said needle having a hollow tube piercing end and a discharge end;
said distal end being pierceable by said needle hollow tube piercing end of said needle to provide for open communication from the inside of said hollow tubular section for transport of any contents of said hollow tubular section through the needle discharge end, wherein said hollow tubular section does not collapse, flatten, or compress longitudinally in an amount that adversely impedes injection of the syringe contents in an amount sufficient to eject more than an insignificant amount of the formulation from the syringe before the entire dose of the syringe is delivered to tissue; and
(ii) an extension piece extending longitudinally from said proximal end of said hollow tubular section and in a direction opposite and away from said hollow tubular section; and
(b) a slideable collar on said extension piece, proximate or adjacent to said proximal end of said hollow compressible tubular section, and external to said hollow tubular section, said collar comprising a compression portion, said compression portion being adapted to slide longitudinally along said longitudinal flanges and the outer surface of said hollow tubular section to compress inwardly said hollow tubular section so that when a needle containing piece is piercing said distal end of said hollow tubular section, at least a portion of any contents of said hollow tubular section passes through said needle discharge end.

2. A syringe as defined in claim 1, wherein said extension piece includes a finger holder for securing a finger on, in, or on and in said extension piece, said finger holder located between the proximal end of the extension piece and the proximal end of said tubular section.

3. A syringe as defined in claim 2, wherein said finger holder comprises a hole extending through said extension piece.

4. A syringe as defined in claim 2, wherein said finger holder comprises at least one arm extending in a direction other than the length of said extension piece.

5. A syringe as defined in claim 1, wherein said slideable collar is attached to said syringe through a breakable joint that is adapted to break said attachment by pressure applied to said slide.

6. A syringe as defined in claim 5, wherein said joint comprises a perforation.

7. A syringe as defined in claim 2, wherein said slideable collar further comprises one or more arms extending in the longitudinal direction of said extension piece and away from said hollow tubular section.

8. A syringe as defined in claim 7, wherein said slideable collar comprises two or more of said arms and at least two of said arms are joined together.

9. A syringe as defined in claim 8, wherein said arms are joined by a bridge.

10. A syringe as defined in claim 1, wherein said hollow tubular section distal end is adapted to receive and secure said needle containing piece through pressure fitting.

11. A syringe as defined in claim 1, wherein said hollow tubular section distal end is adapted to receive and secure said needle containing piece through screw threading.

12. A syringe as defined in claim 1, wherein said hollow tubular section distal end is adapted to receive and secure said needle containing piece through a detent.

13. A syringe as defined in claim 1, wherein said hollow tubular section distal end is adapted to receive and secure said needle containing piece by an annular bulge that fits into a receiving groove in said needle containing piece.

14. A syringe as defined in claim 1, wherein said hollow tubular section distal end is adapted to receive and secure said needle containing piece by groove that receives an annular bulge in said needle containing piece.

15. A syringe as defined in claim 1 which is one-piece.

16. A syringe as defined in claim 1, wherein said tubular section is multi-chambered with a breakable barrier between each chamber.

17. A syringe as defined in claim 1, further comprising a liquid or gel formulation contained in said tubular section.

18. A syringe as defined in claim 1, wherein said collar is a closed collar.

19. A syringe as defined in claim 1, wherein said collar is an open collar.

20. A syringe as defined in claim 1, wherein at least one partial portion of the longitudinal section of said hollow tubular section is flat.
